(19) United States
(12) Patent Application Publication
(10) Pub. No.: US 2006/0041033 A1
(43) Pub. Date: Feb. 23, 2006

(54) Injectable bone-replacement mixture

(76) Inventors: Adrian Bisig, Davos (CH); Marc Bohner, Aarau (CH); Erich Schneider, Davos (CH)

Correspondence Address: JONES DAY, 222 EAST 41ST STREET, NEW YORK, NY, 10017-6702, US

(21) Appl. No.: 11/202,703

(22) Filed: Aug. 12, 2005

Related U.S. Application Data
(63) Continuation of application No. PCT/CH03/00105, filed on Feb. 13, 2003.

(51) Int. Cl. A61K 6/08

(52) U.S. Cl. 523/116

(57) ABSTRACT

An injectable mixture for substituting bone tissue in situ comprises: A) a two-component powder/liquid bone cement which upon mixing forms a self-hardening cement paste; and B) a third component consisting of a liquid which essentially is non-miscible with the cement paste and which is suitable to be washed out after hardening of said mixture in situ, resulting in a porous bone substituting material; and C) an X-ray contrast agent which is an organic substance. The injectable bone substitute material for bone augmentation has adaptable mechanical properties, an optimal radio-opacity without any inorganic X-ray contrast agent and therefore good biocompatibility.

### CROSS REFERENCE TO RELATED APPLICATION

This application is a continuation application of International Application No. PCT/CH2003/000105, filed Feb. 13, 2003, which is incorporated herein by reference in its entirety.

### FIELD OF THE INVENTION

The invention relates to an injectable mixture for substituting bone tissue in situ, in particular for bone augmentation, such as vertebroplasty, femoroplasty (femoral neck augmentation), and humeroplasty (humerus head augmentation).

### BACKGROUND OF THE INVENTION

Polymethylmethacrylate (PMMA) bone cement is by far the most frequently used material known in the art of bone augmentation (e.g., percutaneous vertebroplasty). However, there are serious complications in the use of this material such as cement leakage, monomer toxicity, necrosis, and increased fracture rate of the adjacent vertebrae.

By “cement leakage” is meant the leakage of the injected cement paste out of the bone, in particular into the spinal canal, which can provoke neurological damages such as paralysis. The injected cement can also go into blood vessels and provoke an embolism.

As is well known, PMMA cement hardens according to a very exothermic reaction. Therefore, the tissues surrounding the injected cement might become heated up at temperatures high enough to provoke tissue necrosis.

The increased fracture rate mentioned above is caused by an inadequate stiffness of the augmented segment within an osteoporotic spine and results from the fact that PMMA cement is much stiffer than cancellous bone. Therefore, the whole biomechanical stability of the vertebrae is modified by the presence of the PMMA cement. These biomechanical changes lead to an increased incidence of fractures of the vertebrae adjacent to the augmented vertebrae. The possible countermeasure of prophylactic augmentation of the adjacent levels has the drawback that it enlarges the intervention and enhances the risk for additional cement leakage.

From U.S. Pat. No. 4,093,576 to deWijn it is known to mix a doughy bone cement mixture with a highly viscous aqueous gel to form a dispersion of the bone cement with the gel. This mixture is used for anchoring prosthetic joints into bone, namely to increase the bone soluble, it will be washed out after implantation in the body leaving back a porous bone cement. One of the major drawbacks of the material according to U.S. Pat. No. 4,093,576 deWijn is the use of metallic ions as X-ray contrasting agent. Such particles are incorporated into the gel and therefore, these particles are washed away and can provoke compatibility problems.

In the materials according to the state of the art which use inorganic X-ray contrast agent, like zirconium dioxide and barium sulfate in solid particle form there is a phase separation between the MMA and the inorganic, solid X-ray contrast agent. This is probably caused because of the hydrophilic properties of the heavy metal ions in combination with the hydrophobic properties of the PMMA. If water is used as a third component in the mixture the inorganic X-ray contrast agent selectively accumulates into the aqueous phase. Therefore, complications may occur for clinical applications because of the washing-out of the aqueous phase. Clinical follow-up is not possible because of the lack of radio-opacity after a certain time of washing-out.

Inorganic X-ray contrast agents (BaSO₄, Zr0₂) selectively accumulate into the aqueous phase and thus are washed-out into the blood circulation within a few days with the risk of embolism and toxic reactions. In this context it has to be observed that the amount of X-ray contrast agent necessary for the injection control in bone augmentation is very large, i.e., much larger than for other applications such as the fixation of hip prosthesis (see, for example, U.S. Pat. No. 4,093,576 to deWijn). Washing out of such a large portion of inorganic heavy metal ions in the patient may be very dangerous or even perilous.

### SUMMARY OF THE INVENTION

On this point, the invention intends to provide remedial measures. The invention is based on the objective of providing an injectable self-hardening mixture which upon hardening a subsequent washing out of material in situ results in a porous bone substitute material having a reduced stiffness compared to a conventional hardened PMMA bone cement and which has an optimal radio-opacity.

The invention solves the posed problem with an injectable mixture for substituting bone tissue in situ and the use of and method for preparing such injectable mixture as described below.

The injectable mixture comprises: (a) a two-component powder/liquid bone cement which upon mixing forms a self-hardening cement paste; (b) a third component comprising a liquid which essentially is non-miscible with the cement paste and which is suitable to be washed out after hardening of said mixture in situ, resulting in a porous bone substituting material; and (c) an X-ray contrast agent which is an organic substance.

The invention relates to the use of an injectable bone cement mixture for treating osteoporosis or filling bone defects or the use of the mixture as a carrier for an agent for the treatment of osteoporosis, wherein the bone cement has the property of becoming porous after hardening in situ.

The invention also relates to a method for preparing such an injectable mixture that comprising the following steps: (a) mixing the power and liquid components to obtain a bone cement mixture; and subsequently (b) dispersing the bone cement mixture in the third component.

In another embodiment, the method comprises: (a) mixing the powder and liquid components to obtain a bone cement mixture; and subsequently (b) dispersing the third component in the bone cement mixture.

In yet another embodiment, the method comprises: (a) mixing separately a two-component powder/liquid bone cement; (b) mixing separately a two-component calcium phosphate cement to form the third component; and (c) adding the separately mixed and still pasty two-component calcium phosphate cement to the separately mixed and still pasty two-component bone cement.

The injectable bone substitute material for bone augmentation has adaptable mechanical properties, an optimal radio-opacity without any inorganic X-ray contrast agent and therefore good biocompatibility.

### BRIEF DESCRIPTION OF THE DRAWINGS

FIG. 1 shows the mechanical properties of open-porous cylindrical samples of the hardened mixture according to the invention with different amount of aqueous fraction.

FIG. 2 shows the pore size dependence of the open-porous cylindrical samples of the hardened mixture according to the invention on mixing time of the mixture (top left to bottom right: 30 s, 60 s, 90 s, 120 s).

FIG. 3 shows the mixing time dependency of the mechanical properties of the biphasic cylindrical samples and distilled water with 10 weight % hydroxypropylmethylcellu lose with different amounts of the aqueous fraction, i.e. porosity (P indication the porosity=aqueous fraction) and with different mixing times.

### DETAILED DESCRIPTION OF THE INVENTION

The advantages achieved by the invention are essentially to be seen in the fact that, thanks to the mixture according to the invention:
a) An optimal biocompatibility is achieved by using an organic X-ray contrast agent, preferably one certified for parenteral application. Organic X-ray contrast agents (preferably iodine-containing) can be prepared as an aqueous solution and therefore can directly replace the entire aqueous phase. To allow a follow-up control after washing-out of the aqueous phase preferably a lipophilic X-ray contrast agent can be used additionally with selective accumulation into the PMMA phase.
b) The high radiopacity of the injectable mixture makes it clearly radiologically visible so that cement extravasation during injection can be prevented.
c) The stiffness of the bone substitute material obtained in situ is reduced and adaptable to the properties of osteoporotic bone, hence the risk of fractures of the vertebrae adjacent to the augmented vertebra is lower.
d) A lower amount of polymerization heat is released during polymerization; hence the risk of bone necrosis is lower.
e) The optimal handling and extensive experiences in the application of PMMA based powder/liquid bone cements for vertebroplasty can be utilized.

In a preferred embodiment the X-ray contrast agent is a liquid substance or a solid substance dissolved in a liquid solvent, preferably in water. The X-ray contrast agent may be based on iodine and preferably is chosen from the following group of substances: iopromidum, iopamidol, aminotrizoate acid, iotroxin acid, iopodin acid, iomeprol, iodamid, ioxithalamate, iothalamate, ioxaglin acid and Lipiodol® (iodised ethyl ester of the fatty acids of poppy-seed oil).

The iodine-based X-ray contrast agent may be used in an aqueous solution, preferably in a concentration of 30 to 80 weight %. The injectable mixture may comprises at least 5 weight %, preferably at least 20 weight % of said X-ray contrast agent.

In a further embodiment the viscosity of said third component is lower than 200,000 centipoise. The viscosity of said third component may be lower than 100,000 centipoise, preferably lower than 20,000 centipoise. Typically the viscosity of said third component is 300 centipoise.

In a further embodiment the viscosity of said third component purposefully is comprised between 1,000 and 100,000 centipoise, preferably between 2,000 and 50,000 centipoise.

In a further embodiment the viscosity of the injectable mixture measured 4 minutes after mixing of all components is in the range of 200,000 to 300,000 centipoise. Below 200,000 centipoise the injected mixture tends to leak from the treated bone; above 300,000 centipoise the force required to inject the mixture becomes rapidly too large to enable manual injection.

In a preferred embodiment said two-component bone cement is based on a polyacrylic cement (in particular a polymethacrylic cement) or a calcium phosphate cement. Said two-component bone cement is preferably a powder/liquid system base on polymethylmethacrylate (PMMA) powder and monomethylmethacrylate (MMA) liquid with a polymerization catalyst and a polymerization accelerator.

The third component may comprise water and discrete particles of a water-soluble solid substances. Said water-soluble solid substance may be taken from the group of polysaccharides, in particular: chondroitin sulfate, carboxymethyl cellulose, hydroxyethylmethyl cellulose, fucan, carregeenan, dextran, heparin, heparan sulfate, hydroxyethlycellulose (HEC), hydroxypropylmethyl cellulose, sodium alginate, chitosan or a hyaluronate.

In a further embodiment said third component is an aqueous hyaluronate solution with a concentration of 0.1% to 5.0%, preferably of 1.0% to 2.0%. Typically the concentration may be 0.5%.

The molecular weight of said hyaluronate should be at least 500,000 Daltons, preferably at least 800,000 Daltons. The molecular weight of said hyaluronate should be below 5,000,000 Daltons, preferably below 2,000,000 Daltons. Typically the molecular weight of the hyaluronate used is 1,100,000 Daltons.

Said water-soluble solid substance may be taken from the group of gelatin or collagen. In a further embodiment said third component is a hydrophobic liquid which preferably is selected from the group of: ricinoleic acid (C₁₇H₃₃OCOOH), linoleic acid (C₁₇H₃₁COOH), palmitic acid (C₁₅H₃₁COOH), palmitoleic acid (C₁₅H₂₉COOH), stearic acid (C₁₇H₃₅COOH), linolenic acid (C₁₇H₂₉COOH), arachidic acid (C₁₉H₃₉COOH), myristic acid (C₁₃H₂₇COOH), lauric acid (C₁₁H₂₃COOH), capric acid (C₉H₁₉COOH), caproic acid (C₅H₁₁COOH), oleic acid (C₁₇H₃₃COOH), caprylic acid (C₇H₁₅COOH), erucic acid (C₂₁H₄₁COOH), butyric acid (C₃H₇COOH), ethyl myristate (C₁₃H₂₇COOC₂H₅), ethyl oleate (C₁₇H₃₃COOC₂H₅), ethyl palmitate (C₁₅H₃₁COOC₂H₅), ethyl linoleate (C₁₇H₃₁COOC₂H₅), ethyl laurate (C₁₁H₂₃COOC₂H₅), ethyl linolenate, (C₁₇H₂₉COOC₂H₅), ethyl stearate (C₁₇H₃₅COOC₂H₅), ethyl arachidate (C₁₉H₃₉COOC₂H₅), ethyl caprilate (C₇H₁₅COOC₂H₅), ethyl caprate (C₉H₁₉COOC₂H₅), ethyl caproate (C₅H₁₁COOC₂H₅), ethyl butyrate (C₃H₇COOC₂H₅), triacetin (C₉H₁₄O₆), alpha tocopherol (C₂₉H₅₀O₂), beta tocopherol (C₂₈H₄₈O₂), delta tocopherol (C₂₇H₄₆O₂), gamma tocopherol (C₂₈H₄₈O₂), benzyl alcohol (C₇H₈O), benzyl benzoate (C₁₄H₁₂O₂), methylphenol (C₇H₈O), di-n-butyl sebacate (C₁₈H₃₄O₄), diethylphthalate (C₁₂H₁₄O₄), glyceryl monooleate (C₂₁H₄₀O₄), lecithin, medium chain triglycerides, mineral oil, petrolatum, and liquid paraffines.

In a further embodiment said mixture is divided into a powder component and a liquid component, whereby
A) said powder component comprises the powder component of said two-component bone cement and a polysaccharide in powder form; and
B) said liquid component comprises the liquid component of said two-component bone cement and an aqueous solution of said X-ray contrast agent.

In a further embodiment said third component is a freshly mixed calcium phosphate cement paste.

In a further embodiment the size of all powder particles of said mixture are smaller than 300 micrometers, preferably smaller than 250 micrometers. Purposefully, the size of at least 80% of all powder particles is in the range of 50 to 300 micrometers, preferably in the range of 80 to 250 micrometers. This makes the mixture especially suitable for injection into porous bone structures.

The injectable mixture should harden within 7 to 10 minutes, preferably within 8 to 9 minutes after mixing of its components. This keeps time of anesthesia at a minimum and allows immediate patient weight bearing.

Purposefully the hardened mixture has a Young's modulus of elasticity in the range of 10 to 2800 MPa, preferably in the range of 100 to 700 MPa.

The injectable mixture may further comprise an osteoinductive substance, preferably in its third component. Said osteoinductive substance may be chosen from the following group of substances:
a) bone morphogenetic proteins, preferably BMP2, BMP4 or BMP7;
b) growth factors, preferably TGFb-3 (transforming growth factor) or IGF-1 (insulin-like growth factor);
c) platelet-derived growth factor (PDGF);
d) parathyroid hormone (PHT) and parathyroid hormone-related protein (PTHrP);
e) sexual hormones, in particular estrogen; and
f) prostaglandin.

The injectable mixture may further comprise an antiresorptive substance, preferably in its third component. An antiresorptive substance means a drug, which inhibits resorbtion, i.e., the bone is inhibited to resorb cells. The advantages obtained by the inclusion of such a drug is the possibility of local treatment of osteoporosis which prevents further resorption of the vertebra. Said antiresorptive substance can be a bisphosphonate.

The injectable mixture may further comprise an anabolic substance, a parathyroid hormone (PTH) or an estrogen. An anabolic substance means a drug which generates more bone production, i.e., the bone producing cells are activated.

The injectable mixture may further comprise a hydrogen pump inhibitor, preferably basilomycin Al. The advantage obtained by the inclusion of such a hydrogen pump inhibitor lies in the fact that these drugs are not well applicable systemically and therefore an advantage is obtained by applying them locally.

The injectable bone cement mixture which becomes porous after hardening in situ due to the washing out of its third component is especially useful for treating osteoporosis, for filling bone defects but also as a carrier for an agent for the treatment of osteoporosis.

A possible method for preparing such injectable mixtures for substituting bone tissue in situ may comprise the following steps:
A) the two components of the bone cement are mixed first; and subsequently
B) the obtained mixture is dispersed in the third component.

Another method would comprise the following steps:
A) the two components of the bone cement are mixed first; and subsequently
B) the third component is dispersed in the mixed two-component bone cement.

Still another method would comprise the following steps:
A) Mixing separately a two-component powder/liquid bone cement;
B) Mixing separately a two-component calcium phosphate cement;
C) Adding the separately mixed and still pasty two-component calcium phosphate cement to said separately mixed and still pasty two-component bone cement.

According to a particular embodiment of such methods said third component can be dispersed into the two-component bone cement in such a way that the mean diameter of droplets of the third component dispersed in the two-component bone cement is less than 1 mm, preferably less than 0.5 mm.

The quantity of the injectable mixture to be used for substituting bone tissue in situ depends on the application. In the case of vertebroplasty the quantity is in the range of 2-10 ml. In the case of femoroplasty, the injected volumes are very large, namely up to 40 ml. Especially in this latter application the mixture according to invention has the advantage over conventional materials to exhibit a relatively low temperature rise due to the setting reaction.

The invention and additional configurations of the invention are explained in even more detail with reference to the following examples and having reference to the accompanying figures in which:

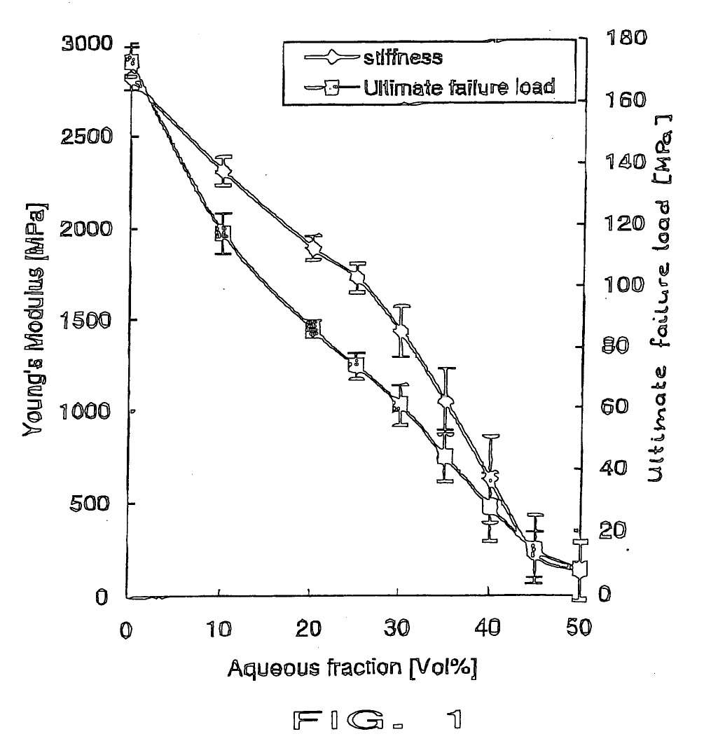

FIG. 1 shows the mechanical properties of open-porous cylindrical samples of the hardened mixture according to the invention with different amount of aqueous fraction.

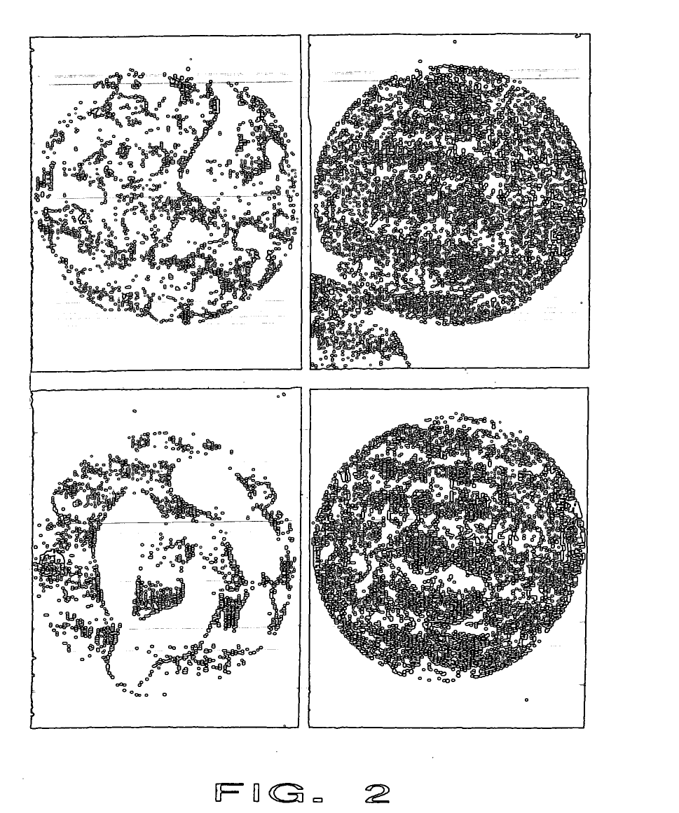

FIG. 2 shows the pore size dependence of the open-porous cylindrical samples of the hardened mixture according to the invention on mixing time of the mixture (top left to bottom right: 30 s, 60 s, 90 s, 120 s).

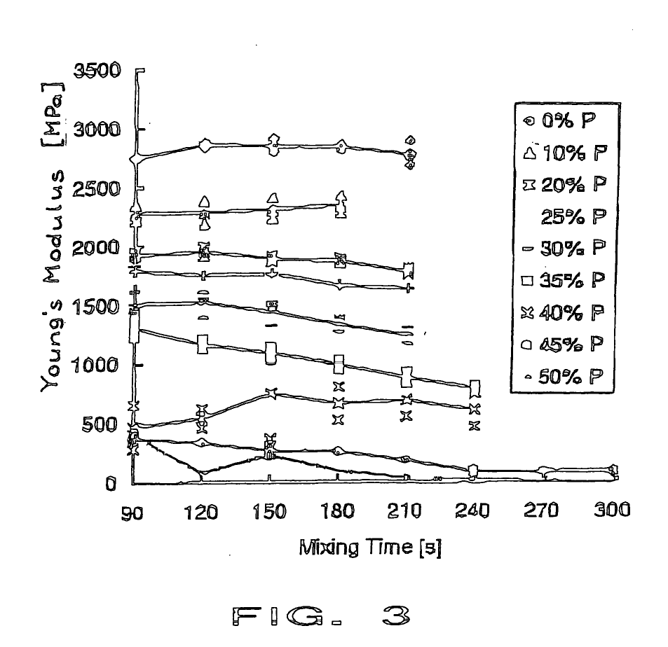

FIG. 3 shows the mixing time dependency of the mechanical properties of the biphasic cylindrical samples and distilled water with 10 weight % hydroxypropylmethylcellu lose with different amounts of the aqueous fraction, i.e. porosity (P indication the porosity=aqueous fraction) and with different mixing times.

### EXAMPLES

### Example 1

### Laboratory

a) Composition of the first component (powder component of the two-component bone cement):
   98.2 weight-% of polymethylmethacrylate (PMMA) as filler
   1.8 weight-% of benzoyl peroxide as polymerization catalyst
b) Composition of second component (liquid component of the two-component bone cement):
   98.0 weight-% of methylmethacrylate (MMA) as curing monomer
   2.0 weight-% of N,N-dimetyl-p-toluidine as polymerization accelerator
c) Composition of third component
   2 weight-% of hyaluronic acid
   98 weight-% of iopromidium as X-ray contrast agent

The porosity of the mixture to be injected is achieved by manual mixing of the highly viscous aqueous fraction represented be the third component to the liquid component (PMMA) of the two-component bone cement. The increased water viscosity is obtained by producing a 2% aqueous solution of hyaluronic acid. The mixing procedure ran in the following manner. Firstly the PMMA powder (powder component of two-component bone cement) and the specific amount of hyaluronic acid (to get a 2% solution) were homogeneously mixed. Then the specific amount of water and—before further mixing—the MMA monomer liquid was added. Manual mixing was done for different durations between 60 s and 150 s to allow more or less spontaneous phase separation between the acrylic and the aqueous phase during the polymerization process. The porosity was assumed to comply with the aqueous fraction.

Cylindrical samples were produced for the mechanical testing of the modified cement. Commercial 2 cc syringes were prepared to serve as cast by cutting off the outflow end. The cement was filled into the syringes by cement injection through a 10 cc syringe. The ‘casting’ syringes were stored vertically during the polymerization for at least 120 min. before pressing out the samples. The environment temperature was 21.5 to 22.0° C. The resulting cylindrical samples had a diameter of 9.54±0.08 mm. The samples were ground within a special adapted steel cast to the length of 16.10±0.09 mm with exactly horizontal tops. The aqueous phase including the whole fraction of the X-ray contrast agent was washed out with water for 60-72 h to achieve an open-porous structure of the hardened cement. The samples were stored into water (22.0° C.) just until the mechanical testing but not longer than one week.

As represented in FIG. 1 the mechanical properties (stiffness measured as Young's Modulus in MPa) and ultimate failure load (measured in MPa) of these samples depend on the amount of the aqueous fraction (in Vol. %).

As shown in FIG. 2 the mixing time importantly influences the pore size. The mixing time influences further the mechanical properties (measured as Young's modulus in MPa) of the hardened material. Several graphs for different degrees of porosity (P in %) are represented.

### Example 2

### Clinical Application

The identical material of example 1 was mixed and 10-15 ml were injected into the lower thoracic spine of a female cadaver. Injectability, radio-opacity and distribution of the biphasic PMMA-water-compound material were comparable to the commonly used PMMA cements (here Vertebroplastice, DePuy). A homogenous distribution of the whole compound without any phase-separation was seen microscopically. Mechanical compression testing of the intact (and cement filled) vertebral bodies showed an increased failure load compared to the non-treated vertebrae. However, the stiffness did not increase in the same amount as for unmodified PMMA cements.

### Example 3

### Laboratory

a) Composition of the first component (first component of the two-component calcium phosphate cement):
   10 g of alpha tricalcium phosphate (Ca₃(PO₄)₂),
   0.5 g of Na₂HPO₄
   4 ml of water
b) Composition of second component (second component of the two-component calcium phosphate cement):
   6.5 g beta tricalcium phosphate (Ca3(PO₄)₂),
   3.5 g monocalcium phosphate monohydrate (Ca(H2PO₄)₂. H₂O)
   0.125 g Na2H2P207, and
   4 ml of a 0.1 M magnesium sulfate solution.
c) Composition of third component
   97 weight-% of Lipiodol® (iodised ethyl ester of the fatty acids of poppy-seed oil)
   3 weight-% of hyaluronic acid

While the foregoing description and drawings are merely illustrative of the principles of the invention, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. In addition, features described herein may be used singularly or in combination with other features. The presently disclosed embodiments are therefore to be considered in all respects as illustrative and not restrictive, the scope of the invention being indicated by the appended claims, and not limited to the foregoing description.

What is claimed is:

1. An injectable mixture for substituting bone tissue in situ, said mixture comprising:
(a) a two-component powder/liquid bone cement which upon mixing forms a self-hardening cement paste;
(b) a third component comprising a liquid which essentially is non-miscible with the cement paste and which is suitable to be washed out after hardening of said mixture in situ, resulting in a porous bone substituting material; and
(c) an X-ray contrast agent which is an organic substance.

2. The injectable mixture of claim 1, wherein the X-ray contrast agent is a liquid substance or a solid substance dissolved in a liquid solvent.

3. The injectable mixture of claim 1, wherein the X-ray contrast agent is based on iodine and is iopromidum, iopamidol, aminotrizoate acid, iotroxin acid, iopodin acid, iomeprol, iodamid, ioxithalamate, iothalamate, ioxaglin acid, or Lipiodol® (iodised ethyl ester of the fatty acids of poppy-seed oil).

4. The injectable mixture of claim 3, wherein the iodine-based X-ray contrast agent is used in an aqueous solution in a concentration of 30 to 80 weight %.

5. The injectable mixture of claim 1, wherein the injectable mixture comprises at least 5 weight % of said X-ray contrast agent.

6. The injectable mixture of claim 1, wherein the viscosity of said third component is less than 200,000 centipoise.

7. The injectable mixture of claim 1, wherein the viscosity of said third component is less than 100,000 centipoise.

8. The injectable mixture of claim 1, wherein the viscosity of said third component is between 1,000 and 100,000 centipoise.

9. The injectable mixture of claim 1, wherein the viscosity of the injectable mixture measured 4 minutes after mixing all of the components is in the range of 200,000 to 300,000 centipoise.

10. The injectable mixture of claim 1, wherein the two-component powder/liquid bone cement is based on a polyacrylic cement or a calcium phosphate cement.

11. The injectable mixture of claim 1, wherein the two-component powder/liquid bone cement is a powder/liquid system based on polymethylmethacrylate (PMMA) powder and monomethylmethacrylate (MMA) liquid with a polymerization catalyst and a polymerization accelerator.

12. The injectable mixture of claim 1, wherein the third component comprises water.

13. The injectable mixture of claim 1, wherein the third component comprises discrete particles of a water-soluble solid substance.

14. The injectable mixture of claim 13, wherein the water-soluble solid substance is gelatin or collagen.

15. The injectable mixture of claim 13, wherein the water-soluble solid substance is a polysaccharide.

16. The injectable mixture of claim 15, wherein the polysaccharide is chondroitin sulfate, carboxymethyl cellulose, hydroxyethylmethylcellulose, fucan, carregeenan, dextran, heparin, heparan sulfate, hydroxyethlycellulose (HEC), hydroxypropylmethyl cellulose, sodium alginate, chitosan or a hyaluronate.

17. The injectable mixture of claim 1, wherein the third component is an aqueous hyaluronate solution.

18. The injectable mixture of claim 17, wherein the aqueous hyaluronate solution has a concentration in the range of 0.1% to 5.0%.

19. The injectable mixture of claim 17, wherein the aqueous hyaluronate solution comprises hyaluronate having a molecular weight of at least 500,000 Daltons.

20. The injectable mixture of claim 17, wherein the aqueous hyaluronate solution comprises hyaluronate having a molecular weight of below 5,000,000 Daltons.

21. The injectable mixture of claim 1, wherein the third component is a hydrophobic liquid.

22. The injectable mixture of claim 21, wherein the hydrophobic liquid is ricinoleic acid (C₁₇H₃₃OCOOH), linoleic acid (C₁₇H₃₁COOH), palmitic acid (C₁₅H₃₁COOH), palmitoleic acid (C₁₅H₂₉COOH), stearic acid (C₁₇H₃₅COOH), linolenic acid (C₁₇H₂₉COOH), arachidic acid (C₁₉H₃₉COOH), myristic acid (C₁₃H₂₇COOH), lauric acid (C₁₁H₂₃COOH), capric acid (C₉H₁₉OH), caproic acid (C₅H₁₁COOH), oleic acid (C₁₇H₃₃COOH), caprylic acid (C₇H₁₅COOH), erucic acid (C₂₁H₄₁COOH), butyric acid (C₃H₇COOH), ethyl myristate (C₁₃H₂₇COOC₂H₅), ethyl oleate (C₁₇H₃₃COOC₂H₅), ethyl palmitate (C₁₅H₃₁COOC₂H₅), ethyl linoleate (C₁₇H₃₁COOC₂H₅), ethyl laurate (C₁₁H₂₃COOC₂H₅), ethyl linolenate, (C₁₇H₂₉COOC₂H₅), ethyl stearate (C₁₇H₃₅COOC₂H₅), ethyl arachidate (C₁₉H₃₉COOC₂H₅), ethyl caprilate (C₇H₅COOC₂H₅), ethyl caprate (C₉H₁₉COOC₂H₅), ethyl caproate (C₅H₁₁COOC₂H₅), ethyl butyrate (C₃H₇COOC₂H₅), triacetin (C₉H₁₄O₆), alpha tocopherol (C₂₉H₅OO₂), beta tocopherol (C₂₈H₄₈O₂), delta tocopherol (C₂₇H₄₆O₂), gamma tocopherol (C₂₈H₄₈O₂), benzyl alcohol (C₇H₈₀), benzyl benzoate (C₁₄H₁₂O₂), methylphenol (C₇H₈₀), di-n-butyl sebacate (C₁₈H₃₄O₄), diethylphthalate (C₁₂H₁₄O₄), glyceryl monooleate (C₂₁H₄₀O₄), lecithin, medium chain triglycerides, mineral oil, petrolatum, or liquid paraffines.

23. The injectable mixture of claim 1, wherein the mixture is divided into a powder component and a liquid component, whereby
(a) said powder component of the mixture comprises the powder component of said two-component bone cement and a polysaccharide in powder form; and
(b) said liquid component of the mixture comprises the liquid component of said two-component bone cement and an aqueous solution of said X-ray contrast agent.

24. The injectable mixture of claim 1, wherein the third component is a freshly mixed calcium phosphate cement paste.

25. The injectable mixture of claim 1, wherein the size of all powder particles of the mixture are smaller than 300 micrometers.

26. The injectable mixture of claim 1, wherein the size of at least 80% of all powder particles is in the range of 50 to 300 micrometers.

27. The injectable mixture of claim 1 which hardens within 7 to 10 minutes after mixing of its components.

28. The injectable mixture of claim 1 which has a Young's modulus of elasticity in the range of 10 to 2800 MPa.

29. The injectable mixture of claim 1, wherein the third component further comprises an osteoinductive substance.

30. The injectable mixture of claim 29, wherein the osteoinductive substance is
(a) bone morphogenetic proteins, preferably BMP2, BMP4 or BMP7;
(b) TGFb-3 (transforming growth factor) or IGF-1 (insulin-like growth factor);
(c) plateled-derived growth factor (PDGF);
(d) parathyroid hormone (PHT) and parathyroid hormone-related protein (PTHrP);
(e) sexual hormones, in particular estrogen; or
(f) prostaglandin.

31. The injectable mixture of claim 1, wherein the third component further comprises an antiresorptive substance.

32. The injectable mixture of claim 31, wherein the antiresorptive substance is a bisphosphonate.

33. The injectable mixture of claim 1, further comprising an anabolic substance, a parathyroid hormone (PTH) or an estrogene.

34. The injectable mixture of claim 1, further comprising a hydrogen pump inhibitor.

35. A method for preparing the injectable mixture of claim 1, comprising the following steps:
(a) mixing the power and liquid components to obtain a bone cement mixture; and subsequently
(b) dispersing the bone cement mixture in the third component.

36. A method for preparing the injectable mixture of claim 1, comprising the following steps:
(a) mixing the powder and liquid components to obtain a bone cement mixture; and subsequently
(b) dispersing the third component in the bone cement mixture.

37. A method for preparing the injectable mixture of claim 1, comprising the following steps:
(a) mixing separately a two-component powder/liquid bone cement;
(b) mixing separately a two-component calcium phosphate cement to form the third component;
(c) adding the separately mixed and still pasty two-component calcium phosphate cement to said separately mixed and still pasty two-component bone cement.

38. The method of claim 36, wherein the third component is dispersed into the two-component bone cement in such a way that the mean diameter of droplets of the third component dispersed in the two-component bone cement is less than 1 mm.

39. The method of claim 37, further comprising dispersing the third component into the two-component bone cement in such a way that the mean diameter of droplets of the third component dispersed in the two-component bone cement is less than 1 mm.
